(19) United States
(12) Patent Application Publication
(10) Pub. No.: US 2006/0231110 A1
(43) Pub. Date: Oct. 19, 2006

(54) Ingestible capsule for esophageal monitoring

(76) Inventor: Martin P. Mintchev, Calgary (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/378,566

(22) Filed: Mar. 17, 2006

Related U.S. Application Data
(60) Provisional application No. 60/664,633, filed on Mar. 24, 2005.

(51) Int. Cl. A61B 19/00; A61N 2/00; A61B 5/05

(52) U.S. Cl. 128/899; 600/350; 600/12; 600/547

(57) ABSTRACT

A capsule containing sensors for impedance and pH monitoring with wireless communication capabilities is presented. A low cost miniature microcontroller interfaces between the sensors and a wireless transmitter. Magnetic holding is proposed as an alternative to surgical affixation of the monitoring capsule. Pins provide additional support for the capsule. The capsule may be used to detect acid and non-acid reflux.

### CROSS-REFERENCE TO RELATED APPLICATIONS

This application claims the benefit under 35 USC 119(e) of provisional patent application No. 60/664,633 filed Mar. 24, 2005.

### BACKGROUND OF THE INVENTION

Gastro-esophageal reflux disease (GERD) is characterized by the reflux of gastric content back into the esophagus. Detection of changes in conductivity, using impedance sensors (MII), and detection of changes in pH using pH sensors, may be used to evaluate non-acidic reflux, acidic reflux, and bolus in the esophagus. While catheters may house the sensors, they cause patient discomfort. Hence, small ingestible pills or capsules, equipped with wireless transmitters, and other sensors, such as temperature sensors and timers, have been developed to locate the sensors in the appropriate position in the esophagus. For example, the Bravo™ capsule, provided by Medtronic Inc., of Shoreview, Minn., is affixable to the mucosal wall of the esophagus using a needle. However, the needle is positioned and removed using an invasive endoscopic clinical procedure. In another example, the Olympus capsule endoscope of Olympus Corporation, Tokyo, Japan, carries an external helix on the capsule. The capsule includes a magnet, and carries video and sampling systems. The capsule is rotated by manipulation of the external magnetic fields, and the external helix propels the capsule through the gastrointestinal system. The Olympus device requires complicated external magnetic field generators. There is therefore room for improvement in existing esophageal diagnostic capsules.

### SUMMARY OF THE INVENTION

According to an aspect of the invention, there is provided an innovative impedance-pH wireless capsule capable of discriminating between acidic and non-acidic reflux, which can be affixed on the inner side of the esophageal wall above the lower esophageal sphincter (LES) and with minimal discomfort to the patient.

According to an aspect of the invention, the esophageal diagnostic sensor may comprise a sensor body having a size and shape suitable for use within the esophagus of a human, a sensor system carried by the sensor body, a processing and communication module connected to the sensor system, and a magnet bound to the sensor body. In operation, a magnetic field generator is disposed about the sensor body, the magnetic field generator being configured to produce a magnetic force on the magnet that is capable of resisting motility forces on the capsule body within the esophagus of a human. According to an aspect of the invention, friction enhancing elements are provided on at least one side of the sensor body to help hold the sensor in the correct position for sensing. The sensor system may comprise impedance sensors and pH sensors.

According to a further aspect of the invention, there is provided a method of sensing one or more conditions of the esophagus, the method comprising the steps of placing a sensor body in the esophagus, where the sensor body is bound to a magnet, a sensor system and a processing and communication module connected to the sensor system, the esophagus having a longitudinal axis, holding the sensor body in the esophagus by frictional forces generated by a magnetic field acting on the magnet established transversely to the longitudinal axis of the esophagus and detecting and analyzing signals sent from the sensor system with a processing and communication module connected to the sensor system.

These and other aspects of the invention are set out in the claims, which are incorporated here by reference.

### BRIEF DESCRIPTION OF THE FIGURES

Preferred embodiments of the invention will now be described with reference to the figures, in which like reference characters denote like elements, by way of example, and in which:

FIG. 1 shows a top view of an embodiment of an esophageal diagnostic capsule according to the invention;

FIG. 2 is a bottom view of the esophageal diagnostic capsule of FIG. 1;

FIG. 3 is a side view of the esophageal diagnostic capsule of FIG. 1;

FIG. 4 is an end view of the esophageal diagnostic capsule of FIG. 1;

FIG. 5 is a circuitry block diagram for a chip used in the esophageal diagnostic capsule of FIG. 1;

FIG. 6 is a flowchart of embedded software used in the esophageal diagnostic capsule of FIG. 1;

FIG. 7 is a diagram showing forces acting on the capsule of FIG. 1;

FIG. 8 shows an embodiment of a vest for ambulatory monitoring using the esophageal diagnostic capsule of FIG. 1;

FIG. 9 shows a further embodiment of an esophageal diagnostic sensor;

FIG. 10 shows a catheter and capsule that may be used together to locate the sensor at a desired position;

FIG. 11 shows an embodiment of an esophageal diagnostic sensor using a flexible element to connect the magnet to the capsule; and

FIG. 12 shows more detail of the embodiment of FIG. 11.

### DETAILED DESCRIPTION OF PREFERRED EMBODIMENTS

In the claims, the word “comprising” is used in its inclusive sense and does not exclude other elements being present. The indefinite article “a” before a claim feature does not exclude more than one of the feature being present.

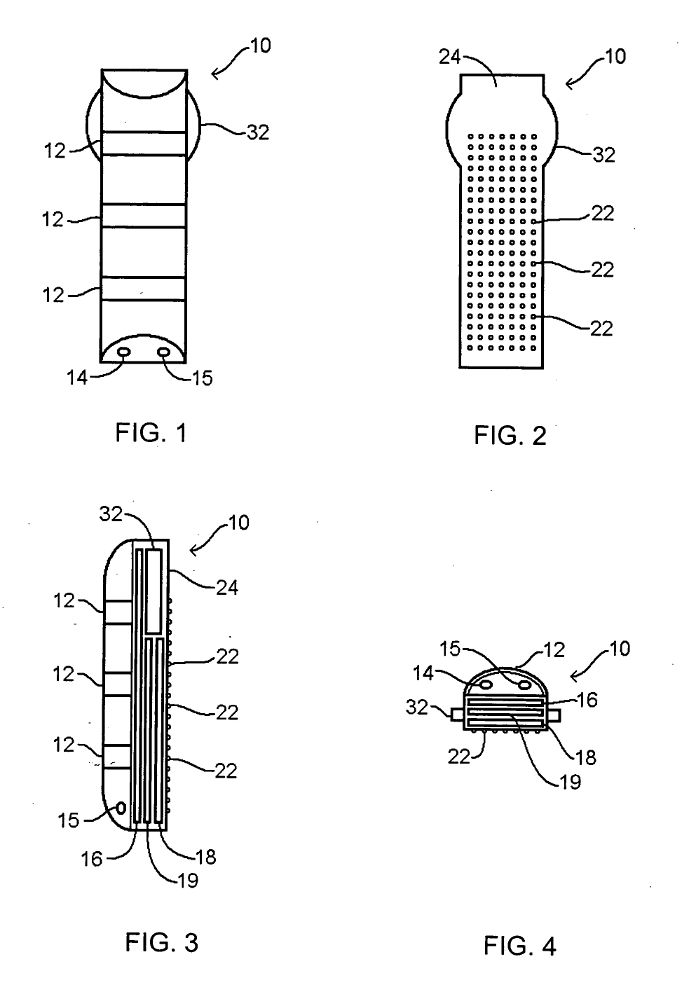

An exemplary esophageal diagnostic sensor is shown in various views in FIGS. 1-4. The sensor has sensor or capsule body 10 having a size and shape suitable for use within the esophagus of a human. A sensor system is carried by the sensor body 10. In the example presented, the sensor system comprises three half-ring impedance electrodes 12 separated from each other longitudinally and each being distributed circumferentially around the sensor body 10. The sensor system also in this example comprises a pH sensor 14, 15 formed of a sensing electrode 14 and reference electrode 15. Stainless steel half-ring electrodes 12 may be utilized to measure impedance and discriminate between acidic and non-acidic esophageal reflux. pH sensor electrodes 14, 15 may comprise a standard antimony electrode 14 linked via electronics to an internal reference electrode 15. The sensor system may also include any number of other sensors suitable for detecting a physiological parameter of the esophagus, or any other part of the gastrointestinal tract of a human, including video cameras and pressure sensors. A processing and communication module 16 is connected to the sensor system 12, 14. A magnet 18 is bound to the sensor body 10 by in this exemplary case being contained within the sensor body 10 and separated from the processing and communication module 16 by insulating spacer 19. The magnet 18 may for example be a neodymium-iron-boron magnet of suitable size and shape, for example a 6.5 mm×26 mm×1.5 mm magnet, included in the capsule to facilitate magnetic holding.

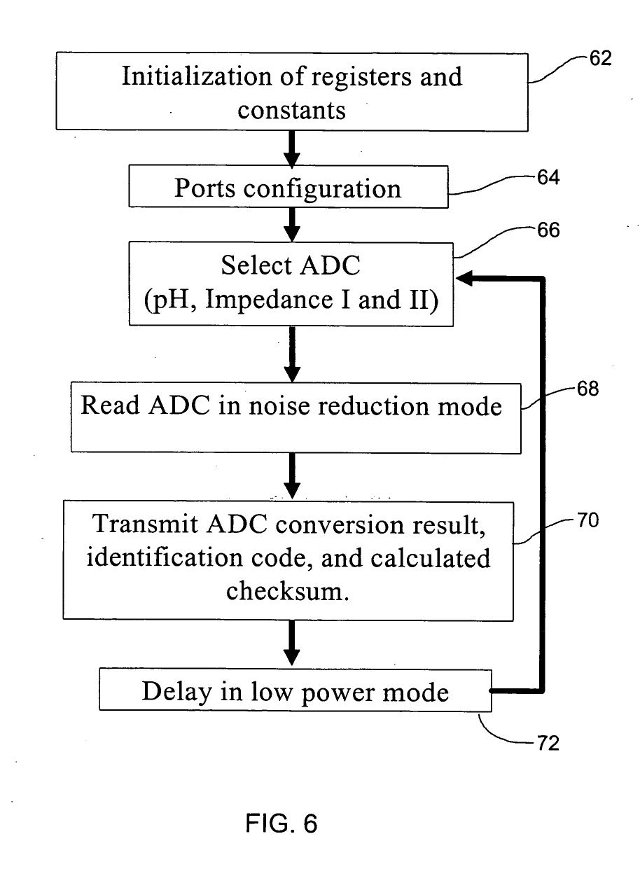

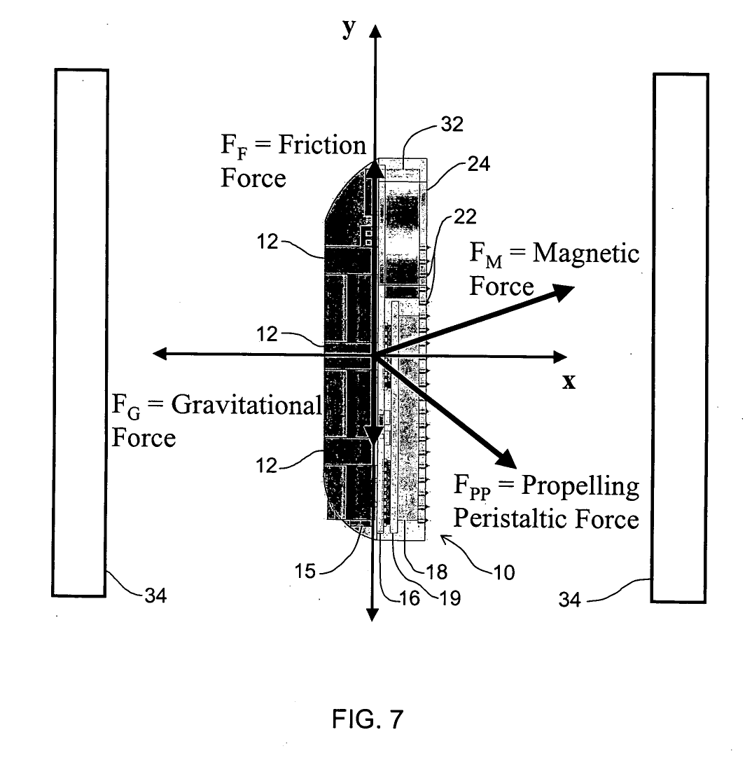

As shown in FIGS. 6 and 7, a magnetic field generator 20 is disposed about the sensor body 10. The magnetic field generator 20 produces a magnetic force on the magnet 18 that is capable of resisting motility forces on the sensor body 10 within the esophagus of a human. The sensor body 10 also is preferably provided with friction enhancing elements, which in the example shown are pins 22 protruding from one side 24 of the sensor body. The friction-enhancing pins 22 increase the static friction coefficient between the capsule and the mucosal wall of the esophagus to aid magnetic holding.

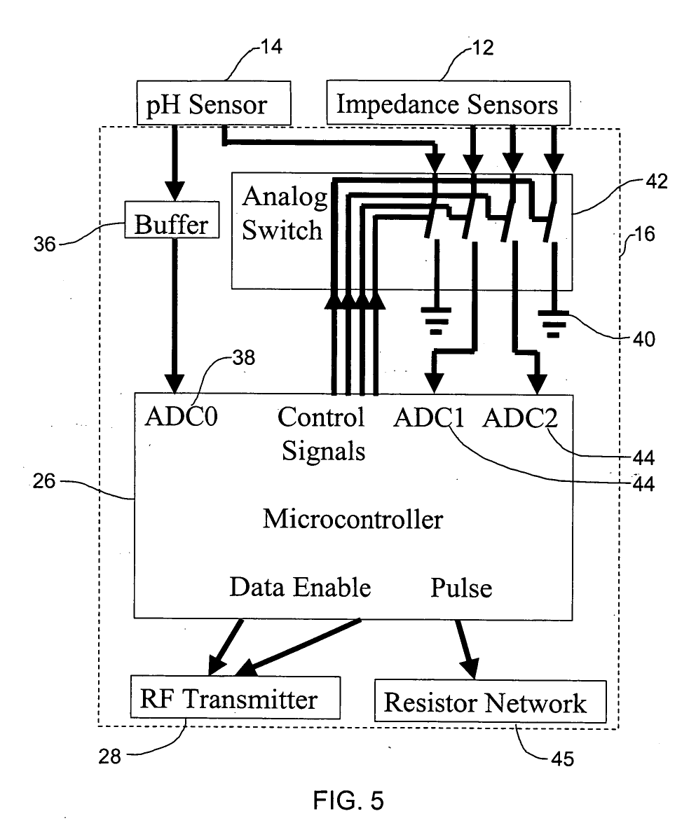

As shown in FIG. 5, the processing and communication module 16 may be formed by a miniature microcontroller 26 used to perform analog-to-digital (A/D) conversion of the data, power management, and wireless communication control. An example microcontroller 26 may be an Attiny26™ microcontroller, available from Atmel Corporation, San Jose, Calif. The processing and communication module 16 also includes in this example a radio frequency (RF) transmitter 28 linked by conventional communication links, as for example, wires, indicated, as with other communication links in FIG. 5, by simple lines. The RF transmitter 28 provides wireless communication between the capsule 10 and a receiving computer station, which could be a portable decoder 30 as shown in FIG. 7, or may be any conventional computer such as a MAX1472, available from Maxim Integrated Products Inc., Sunnyvale, Calif. A battery 32 (FIGS. 1, 2 and 3), for example a 9 mm, 30 mAh 3 volt CR927™ battery available from Tian Qiu Corporation, China supplies power to the module 16.

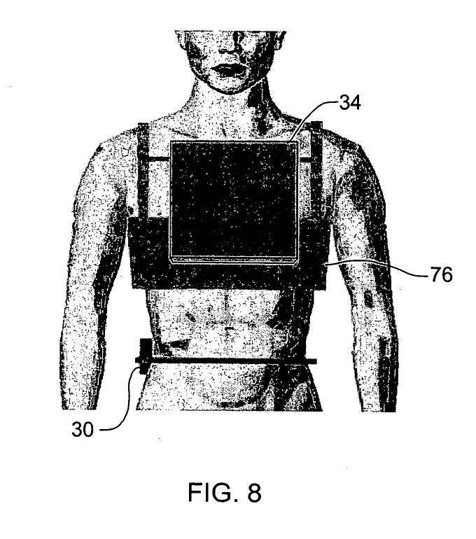

The sensor body 10 itself may be a 28 mm×8 mm×8 mm capsule. The capsule 10 is held in position using a magnetic field generator, such as may be produced by magnets or coils 34 disposed about a patient's body, as illustrated in FIGS. 7 and 8. The magnets 34 may be for example two 10 cm×10 cm×5 cm, 5000 Gauss, square magnets positioned 27 cm apart or any other suitable magnets.

The pH sensor may comprise an antimony electrode 14 and a reference electrode 15. The antimony electrode 14 may be formed of an ingot of metallic antimony, for example 99.999% metal basis (Alfa Aesar, Ward Hill, Mass.), pulverized, melted and chemically treated to form antimony billets of 1 mm in diameter and 2 mm in length. Reference electrode 15 may for example be formed of a silver wire (California Fine Wire, Grover Beach, Calif.), chloridized, rinsed and baked at 175° C. The antimony billet is soldered to a copper wire connected to the module 16. The reference electrode 15 may be coated in gel (Signa Gel, Parker Laboratories, Inc., Fairfield, N.J.) and encapsulated next to the antimony electrode 14. The fabricated pH sensor may have for example dimensions of 2.2 mm in diameter by 20 mm in length. The difference in voltage between the reference electrode 15 and the antimony billet 14 depends on the pH value of liquids lying in contact with the electrodes 14, 15. A miniature low power operational amplifier (for example an LMV981BL, National Semiconductors Corporation, Santa Clara, Calif.) may be used as a buffer 36 between the pH sensing electrodes 14, 15 and an analog-digital converter 38 in the microcontroller 26. The reference electrode 15 may be connected to ground 40 through a switch 42 such as a 14-Quad-Flatpack-No-Lead analog switch available from Texas Instruments Inc., Dallas Tex.) to minimize interference provoked by the impedance stimulating pulse.

The impedance sensors 12 may be made using stainless steel half-ring electrodes, split ring electrodes or any other suitable electrodes. The electrodes 12 are driven by five cycles of a 100-Hz square pulse from the microcontroller 26. A resistor, such as a 100 kΩ resistor network 45, may be used to limit the current flow through each of the electrodes 12. The sensing electrodes 12 and the reference may be connected to the microcontroller 26 and ground 40 through the analog switch 42 and AD converters 44. Two impedance channels may for example be implemented using three 2.5 mm wide half-ring electrodes 12. The distance between the electrodes depends on the application, but may be in the order of 2.5 mm.

Voltage changes in the pH and impedance sensors are monitored by the A/D converters 38, 44 of the microcontroller 26. Digital data from the A/D converters 38, 44 may be stored in memory, and periodically transmitted from the capsule 10 using the transmitter 28. Control software for the microcontroller 26 may be designed using the description in this patent document. Security identifications may be added to each reading. To assure the integrity of the data during the wireless communication, a checksum test may be implemented. The microcontroller 26 may also determine the delays between transmissions in order to save power and generated all the necessary control signals. A flowchart diagram of the embedded microcontroller software is shown in FIG. 6. The process begins with initialization of registers and constants in step 62 and ports configuration in step 64. Next, step 66, the analog-digital converters 38 or 44 are selected depending on which sensor input is to be read. The microcontroller 26 then, step 68, reads the chosen analog-digital converter, preferably in noise reduction mode if available. The converted readings are transmitted in step 70 to the receiving computer station 30, with identification code and checksum, if used. Delays between transmissions are provided in step 72 and these and security identifications can be easily programmed before assembling the capsule 10. Thus, these parameters can be optimized according to the needs of the user. Frequency encoding may be implemented for the wireless communication.

Outside the body, a receiving computer station 30 receives the transmitted signals. For example, a MAX1473 receiver kit (Maxim Integrated Products Inc., Sunnyvale, Calif.) may be used. A microcontroller may also form part of the receiver kit, for example an Attiny26 microcontroller. The received digital results are acquired into a computer forming part of the computer station 30, which may be a conventional computer, and converted to pH and impedance values for real-time monitoring, data analysis and system debugging. A DAQCard AI-16XE-50 and C for Virtual Instrumentation (CVI) v. 7.0.0 may be used to implement this stage (National Instruments Corporation, Austin Tex.).

The sensor uses a magnetic field to fix the position of the sensor in the body. For example a field of 1000 Gauss is sufficient to hold a 20 g mass in position against gravity. The human esophagus is located approximately at the center of the rib cage, behind the lungs and in front of the spinal cord and the aorta. The magnetic force holding the capsule in place can be combined with static friction between the shell of the capsule and the inner side of the esophageal wall to overcome the propelling peristaltic force (F_{PP}). The static friction is defined as the required force to start moving a body at rest. The static friction coefficient (μ) between two solid surfaces can be defined as the ratio of the tangential force (F) required to cause the movement, divided by the normal force between the surfaces (F_{N}).
μ=F/F_{N} (1)

The forces acting on the capsule are shown in FIG. 7. The magnitude and the angle of F_{PP} depend on how the peristaltic force is distributed on the surface area of the capsule. F_{PP} is shown at −45° because the shape of the capsule is expected to cause this effect. The normal force F_{N} experienced by the capsule is the resultant of the “x” component of the magnetic force (F_{MX}=F_{M} Cos(θ₁)), plus the “x” component of F_{PP} (F_{PPX}=F_{PP} Cos(θ₂)). The “y” component of the magnetic force is expected to overcome gravity. The “y” component of F_{PP} (i.e. F_{PPY}=F_{PP} Sin(θ₂)) corresponds to F in Eq. 1. Therefore, the required static friction coefficient to hold the capsule against peristalsis can be determined as:
μ=F_{PPY}/(F_{MX}+F_{PPX}) (2)

Manometric recordings obtained on healthy volunteers during peristalsis show that the strongest pressure (P) provoked by peristalsis is about 96 mmHg during meals. This corresponds to 12.8 kPa or 12800 Newtons per square meter. The force propelling the capsule depends on the shape of the capsule and the angle between the surface of the capsule and the contracting esophageal wall at the point of contact. Therefore, a capsule housing design at an angle ≧45° considerably reduces the propelling force acting on its surface. Peristaltic forces acting at a 90° angle would not propel the capsule, but instead would only push it against the esophageal wall. Therefore, the surface area A of the capsule exposed to F_{PP} is only at the proximal end. If we consider this area to be approximately ¼ of the capsule total surface area, and at a 45° angle, F_{PPY} can be estimated as:
F_{PPY}=(P*A/4)sin(−45°)=−0.863 N. (3)

A force of 0.863 N corresponds to a mass of 88 g against gravity. The negative sign indicates the direction of the force. Therefore, a combination of the external magnetic field and friction-enhancing pins capable of handling a load of about 100 g without penetrating the mucosa should be able to overcome F_{PPY}. An array of 18 stainless steal pins 22 (0.16 mm in diameter and 0.7 mm in length) may for example be built by silver-soldering the pins to a stainless steel plate (Stay-Brite Silver Solder Kit, J. W. Harris Corporation, Manson, Ohio). The inventors have found that the combination of the magnetic field and the friction enhancing elements is sufficient to hold the capsule 10 in position. That is, F_{M} is strong enough to cancel F_{G}. The friction-enhancing pins are able to hold a 100 g load against gravity. Therefore, it is believed that the capsule 10 can overcome esophageal peristalsis while remaining affixed to the esophageal wall without penetrating the mucosal lining.

A capsule design for combined impedance-pH monitoring has been presented. In contrast to previously proposed solutions, this design is able to discriminate between acidic and non-acidic reflux. Other magnets, preferably lighter magnets, may be used for the magnets. Magnets with dimensions of 20 cm×20 cm×1 cm, which deliver the same magnetic flux, may be used in a vest 76 used for application on humans (FIG. 8).

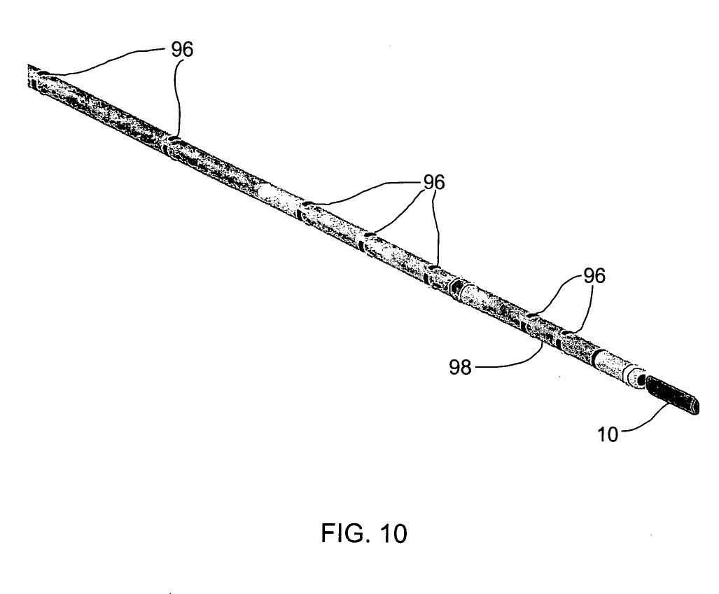

The swallowable capsule 10 for pH monitoring may also include a pressure sensor and a receiving coil embedded in the capsule casing for power transfer instead of a battery. Circumferential split-electrodes may be used for the impedance sensors, which are advantageous due to their very small size. Split ring electrodes 96 are shown in FIG. 10. In a split-ring electrode, reference and sensing electrodes alternate circumferentially around the capsule. Typically, there may be two reference electrodes at opposing sides of the capsule, and two sensing electrodes at opposing sides, thus one electrode for each quadrant around the capsule. If the electrodes are actuated with 3.0 volts through an integrated oscillator, the total power consumption may be around 0.15 mW per channel. The capsule may monitor changes between neutral and acidic pH along the gastrointestinal tract plus any additional phenomena of interest (for example, pressure). The capsule 10 may be affixed 5 cm above the lower esophageal sphincter and if the overall power consumption allows it, a standard pH 24-hour monitoring test could be performed.

In order to provide a meaningful and sufficiently long (preferably, 24-hour or longer) pH monitoring, the capsule has to be affixed at a particular location on the inner side of the esophageal wall (preferably, about 5 cm above the LES). The external magnet may be worn by the patient in a specially designed band, belt or vest, and positioned in such way so that that the internal miniature magnet is attracted to it. The enclosure may contain also a Hall effect sensor, which may be used to quantify the bond between the internal permanent magnet embedded into the capsule, and the cutaneous DC magnet controlling its position, so that a feedback mechanism can be implemented maintaining the force bonding the two magnets to be strong enough to keep the capsule at the desired position overcoming the peristaltic forces, but not substantially strong to displace the wall of the esophagus by moving it closer to the external magnet. The capsule can be power-supplied either by an autonomous battery, or transcutaneously using electromagnetic inductance. The transmitting coil can be located on a belt or a vest worn by the patient, which contains also the external DC magnet for affixing the capsule. The receiving coil may be wrapped around the inner circumference of the capsule in a spiral fashion.

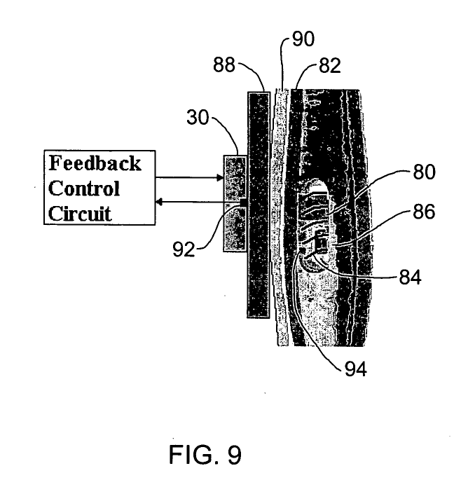

FIG. 9 shows a further embodiment of an esophageal diagnostic sensor according to the invention. In FIG. 9, a capsule 80 is shown pressed to one wall 82 of a human esophagus. A power receiving coil 84 is embedded in the outer shell 86 of the capsule 80. In this embodiment, the location of the capsule 80 is above the LES, and it is affixed there using an external magnetic field provided by a DC magnet 88 mounted in a vest worn by the patient and pressed against the skin 90 of the patient's chest. An exterior Hall-effect sensor 92 linked to a computer station 30 (not shown in FIG. 9, but shown in FIG. 8) provides a feedback mechanism for controlling the strength of the bond between an internal permanent magnet embedded in the capsule 80 and the external DC magnet 88 producing the magnetic field needed to keep the capsule 80 at a desired location. A second Hall effect sensor 94 may be carried by the capsule 80. The Hall effect sensor 94 outputs a signal to a processing and transmitting module carried by the capsule 80, and the signal is transmitted to the computer station 30. By measuring the fields at the two Hall effect sensors, the location of the capsule 80 in relation to the exterior magnet 88 may be determined. Alternatively, the capsule position may be determined using only one of the Hall effect sensors. If the Hall effect sensor carried by the capsule is used for position determination, then the processing and transmitting module 16 may be used to transfer position signals to the computer station 30. Magnetic field strength and configuration may be altered by using coils for the exterior magnets, and adjusting the current in the coils, by moving the magnets, or moving the patient's position in relation to the magnets.

A transmitting coil (not shown) may be used to transfer wireless power to the capsule 80. The frequency of the electromagnetic field for transcutaneous power transfer is high enough (in the MHz range), so that the permanent magnet in the capsule 80 is not influenced by this alternating electromagnetic field, but continues to be controlled by the strong external DC magnet, preserving its affixed position above the LES and having the feedback control mechanism based on the Hall effect sensor.

The capsule 10 or 80 may also be located in the esophagus using a pressure-monitoring catheter 98 shown in FIG. 10. Once the capsule 10 or 80 is located at the proper position as indicated by pressure measurements made by the pressure monitoring catheter, it may be released from the catheter. The capsule 10 or 80 can be released from the catheter using an appropriate eternally-controlled mechanical mechanism, and after the external DC magnet is adjusted to hold the capsule in the correct position. The pressure monitoring catheter may be such as is disclosed in U.S. patent application Ser. No. 11/163,342 filed Oct. 14, 2005, the content of which is incorporated herein by reference, although any suitable pressure sensing catheter may be used.

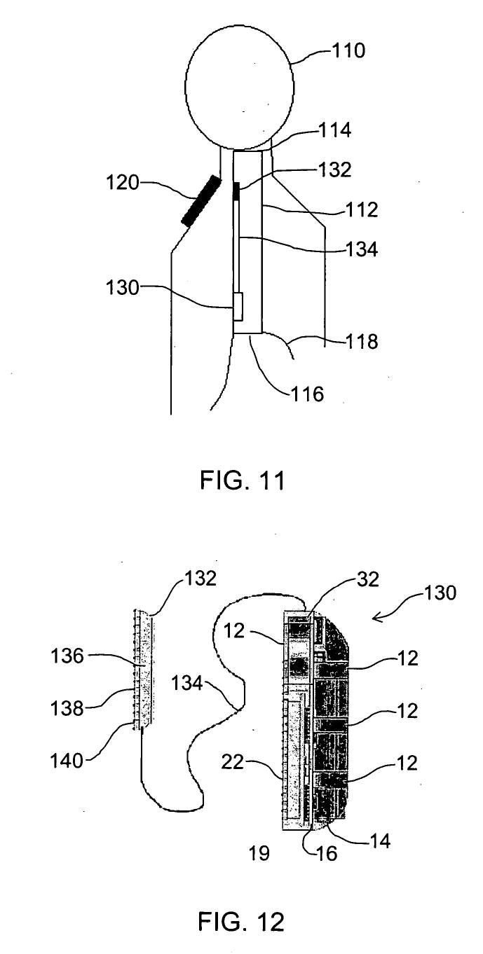

A further embodiment of an esophageal diagnostic sensor will now be described with reference to FIGS. 11 and 12. The human esophagus is a long hollow organ that transports food from the mouth to the stomach. A way to reduce magnet size and weight is to locate the magnet as close as possible to the esophagus. FIG. 11 shows a patient 110, with esophagus 112, upper esophageal sphincter 114, lower esophageal sphincter 116 and stomach 118. The esophagus 112 is evidently closer to the surface of the skin at the base of the neck. The distance between the esophagus 112 and an external magnet 120 for an average adult male at the base of the neck is around ⅓ of the neck diameter. This is approximately less than 5 cm for an adult male. However, this point is about 15 cm away from the LSE 116. Thus a capsule 130 bound to a magnetic holder 132 using a soft flexible thread 134 may be used as shown in FIGS. 11 and 12. Other methods of binding the sensor body to the magnet may be used.

In this embodiment, magnetic holder 132 is made of a suitable plastic or metal, and houses a magnet 136. Friction enhancing devices 138 such as pins protrude from one face 140 of the magnet holder 132. The magnet holder 132 is held in position utilizing one or more magnets or electromagnets 120 located at the base of the neck. The magnet 120 should be able to hold a 6 g mass against gravity at 5 cm from its surface. A magnetic field of 200 gauss has been found to be sufficient to hold a 6 g mass against gravity. The capsule 130 may be of the same construction as the capsule 10, and thus similar reference numerals have been used to identify the various components, but with the magnet 18 omitted. The soft flexible element 134 may be made of any suitable material or construction type, for example chain links, that provides a flexible supporting link between the magnet 136 and capsule 130.

As shown by the analysis of F_{PP} set out above, the combination of the external magnetic field and friction-enhancing elements capable of handling a load of about 100 g without penetrating the mucosal wall of the esophagus should be able to overcome F_{PPY}. An exemplary friction enhancing element is an array of 18 stainless steel pins (0.16 mm in diameter and 0.7 mm in length) silver-soldered to a stainless steel plate. A permanent neodymium magnet 120 of 5 cm×4 cm×2.5 cm for example will generate the desired magnetic field at 5 cm from its surface. An electromagnet able to generate a similar field may also be used. For example, with a current of 3 amperes, 1000 turns would be required to generate the desired field. Both electromagnets and permanent magnets are able to hold a capsule in position. However, electromagnets are larger in size and require a power supply able to continuously deliver 3 A. On the other hand, permanent magnets can be of a smaller size and do not require a power supply. The main disadvantage of permanent magnets is the lack of control on the magnetic field.

An innovative multi-sensor esophageal capsule design has been presented for the purpose of simultaneous detection of acidic and non-acidic gastro-esophageal reflux. The obtained results suggest that if appropriate shielding of the external permanent magnets is provided, and the latter are of appropriate weight and size, this technique may offer a minimally invasive and reliable testing of all aspects of GERD. Immaterial modifications may be made to the embodiment of the invention disclosed without departing from the invention.

What is claimed is:

1. An esophageal diagnostic sensor, comprising:
a sensor body having a size and shape suitable for use within the esophagus of a human;
a sensor system carried by the sensor body;
a processing and communication module connected to the sensor system;
a magnet bound to the sensor body; and
friction enhancing elements on at least one side of the sensor body.

2. The esophageal diagnostic sensor of claim 1 further comprising a magnetic field generator disposed about the sensor body, the magnetic field generator being configured to produce a magnetic force on the magnet that is capable of resisting motility forces on the capsule body within the esophagus of a human.

3. The esophageal diagnostic sensor of claim 1 in which the sensor system comprises impedance sensors and pH sensors.

4. The esophageal diagnostic sensor of claim 4 in which the friction enhancing elements comprise pins.

5. The esophageal diagnostic sensor of claim 1 in which the magnet is bound to the sensor body by a flexible link.

6. An esophageal diagnostic sensor, comprising:
a sensor body having a size and shape suitable for use within the esophagus of a human;
a sensor system carried by the sensor body, the sensor system comprising impedance sensors and pH sensors;
a processing and communication module connected to the sensor system;
a magnet bound to the sensor body; and
friction enhancing elements on at least one side of the sensor body.

7. The esophageal diagnostic sensor of claim 6 further comprising a magnetic field generator disposed about the sensor body, the magnetic field generator being configured to produce a magnetic force on the magnet that is capable of resisting motility forces on the capsule body within the esophagus of a human.

8. A method of sensing one or more conditions of the esophagus, the method comprising the steps of:
placing a sensor body in the esophagus, where the sensor body is bound to a magnet, a sensor system and a processing and communication module connected to the sensor system, the esophagus having a longitudinal axis;
holding the sensor body in the esophagus by frictional forces generated by a magnetic field established transversely to the longitudinal axis of the esophagus and
detecting and analyzing signals sent from the sensor system with a processing and communication module connected to the sensor system.

9. The method of claim 8 in which the sensor system comprises impedance sensors and pH sensors.

10. The method of claim 8 further comprising the step of enhancing the frictional forces by providing friction enhancing elements on at least one side of the sensor body.

11. The method of claim 10 in which the friction enhancing elements comprise pins.

12. The method of claim 8 further comprising adjusting the magnetic field to move the capsule within the patient's gastrointestinal tract.

13. Apparatus for monitoring one or more physiological characteristics or parameters in a patient's gastrointestinal tract, the apparatus comprising:
a magnetic field generator positioned outside the patient's body for producing a magnetic field inside the person's gastrointestinal tract;
a capsule linked to magnetic material of sufficient mass under influence of the magnetic field to hold the capsule in a desired position against motility forces in the patient's gastrointestinal tract;
at least one sensor carried by the capsule that is capable of sensing a physiological condition or parameter in the gastrointestinal tract; and
a Hall effect sensor responsive to the position of the capsule to produce position signals representing the position of the capsule.

14. The apparatus of claim 13 in which the Hall effect sensor is carried by the capsule.

15. The apparatus of claim 14 further comprising a second Hall Effect sensor outside of the body, the controller being responsive to signals from both the Hall Effect sensor in the capsule and the second Hall Effect sensor to determine the position of the capsule.
